(19) United States
(12) Patent Application Publication
(10) Pub. No.: US 2022/0022933 A1
(43) Pub. Date: Jan. 27, 2022

(54) Plasma Probe with Improved Ignition Behavior

(71) Applicant: Erbe Elektromedizin GmbH, Tuebingen (DE)

(72) Inventor: Klaus Fischer, Nagold (DE)

(21) Appl. No.: 17/375,487

(22) Filed: Jul. 14, 2021

(51) Int. Cl. A61B 18/04; A61B 18/08

(52) U.S. Cl. CPC A61B 18/042; A61B 2018/00589; A61B 18/082

(57) ABSTRACT

A plasma probe (10) comprises a fluid line (14) that is preferably configured as a hose and an electrode (17) arranged therein, the tip (20) of which is located in the proximity of the outlet opening (16). The electrode (17) comprises a hydrophobically configured surface section (21). As an option, additional elements of the plasma probe (10), particularly in the proximity of the outlet opening (16), can be provided with hydrophobic surfaces. Such a probe comprises a remarkably improved ignition readiness and an improved steadiness due to the permanence of the obtained discharge base point.

### RELATED APPLICATION(S)

This application claims the benefit of European Patent Application No. 20187417.9, filed Jul. 23, 2020, the contents of which are incorporated herein by reference as if fully rewritten herein.

### TECHNICAL FIELD

The invention refers to a plasma probe, particularly for plasma coagulation of biological tissue. Further the invention particularly refers to a plasma probe for endoscopic use.

### BACKGROUND

A plasma probe is known from DE 69 636 399 T2 that consists of a tube made of a bendable material. This tube is guided through a working channel of an endoscope in the body of a patient. The tube comprises an end piece that is made of temperature-resistant material, such as PTFE or ceramic. Similarly the tube can consist of PTFE (Polytetrafluorethylene). An electrode is arranged in the channel enclosed by the tube and the end piece, the tip of which does not project out of the opening of the tube or the end piece. The electrode is connected to a voltage source. Inert gas, for example argon, flows through the channel enclosed by the tube such that a plasma jet is output from the probe. Thereby a regulator unit allows the adjustment of the volume flow of gas that can at least provide two different amounts of gas flows. In the standby operation the gas flow is low. In order to guarantee that fluids entering the probe are ejected from the probe during plasma ignition, the gas stream is increased during operation. In doing so, it can be avoided that blood residues accumulate in the tube, which would cause the danger of infection during a later surgery on another patient (compare paragraph [0041]).

Another plasma probe is known from DE 100 30 111 B4 that consists of a thin hose with a heat-resistant distal end piece and an electrode arranged in the lumen of the hose. The electrode is particularly formed by a sheet metal provided with distal tip, the longitudinal edges of which are supported at the inner side of the hose.

A plasma probe with a hose made of PTFE or a similar material is known from EP 0 957 793 B2. This hose is provided with an end piece at the distal end that consists of PTFE or ceramic. An electrode is arranged in the hose around which noble gas flows that is channeled by the hose and that forms a plasma jet. Again the gas flow can be reduced in standby phases and can be increased during activation in order to avoid the ingress of body liquids and displacement thereof to other patients by means of the probe.

In addition, EP 0 957 793 B2 proposes a plasma probe, the end piece of which consists of ceramic. A tube-shaped electrode is directly connected to the proximal end of the hollow shank of the ceramic end piece, whereby the inner diameter of the electrode corresponds to the inner diameter of the ceramic end piece. This arrangement serves for coagulation of larger tissue areas.

In practical operation the probes described above occasionally show a bad ignition behavior. This means that for the formation of a plasma discharge either particularly high voltages have to be used or that the probe has to be moved closely to the tissue to be treated.

Starting therefrom it is an object of the invention to provide a plasma probe with improved ignition behavior.

### SUMMARY

This object is solved by a plasma probe as described herein.

The inventive plasma probe in one form comprises a flexible fluid line that can be formed by a hose made of a suitable plastic material, such as for example, PTFE or another suitable plastic. The fluid line limits at least one channel. The proximal end thereof can be connected to a gas source. The gas source serves for supplying a defined gas flow to the channel that exits an outlet opening formed at the distal end of the fluid line.

In addition, the plasma probe comprises an electrode that can be connected to an electrical source via an electrical line. The line can be a wire or a strand or the like extending through the channel or a conductor guided on or in the wall of the hose. The electrical source can be a radio frequency generator, as it is provided in medical apparatus for supply of such probes. The electrode is preferably arranged in proximity to the outlet opening. The end of the electrode that is preferably formed in a pointed manner is preferably arranged such that it does not project out of the outlet opening. In doing so, harm of the biological tissue due to a mechanic influence of the electrode can be avoided. As an alternative, the electrode can extend through the outlet opening and can carry a blunt body at the end that is preferably electrically insulating in order to avoid injuries of tissue. Such a probe is suitable for the omnidirectional lateral plasma output.

According to one aspect of the invention, a hydrophobic surface section is formed on the electrode. Preferably this refers particularly to a section of the electrode that is nearby the distal end of the electrode or that adjoins the distal end of the electrode. The surface of the electrode can also be hydrophobically configured as a whole.

Due to the hydrophobic configuration of the surface of the electrode its ignition behavior is remarkably improved. If during the introduction of the plasma probe in the endoscope liquids, e.g. flushing liquid, wet the plasma probe, such liquids tend to ingress in the open outlet opening of the plasma probe. Such liquid droplets can influence the gas flow, can create turbulences and asymmetries in the gas flow and can hinder the electron emission at the tip of the electrode. If the electrode, however, is water-repellant (hydrophobic), such effects are reduced or avoided.

The hydrophobic surface section of the electrode also avoids that biological tissue, tissue liquid or products coming from the biological tissue accumulate on the electrode and modify its surface conductivity during the operation of the plasma probe. In doing so, also a movement of the discharge base point and particularly a movement of the discharge base point away from the electrode tip into the channel of the probe is prevented. In doing so, also the otherwise imminent thermal overload of the probe is avoided. Such an excessive thermal strain could otherwise also result in a deformation of the fluid line, which in turn would massively hinder the gas flow and the creation of a plasma stream. Also a direct contact between the electrode and the tissue could occur in case of a respective probe damage. The consequences of a moving discharge base point are largely or completely avoided.

The water-repellant configuration of the electrode, particularly of the surface section thereof near the tip in addition results in an increased ignition readiness, particularly in adverse, i.e. particularly wet conditions of use.

The inventive plasma probe can comprise a fluid line that itself consists of heat-resistant material or that is provided with a heat-resistant end piece at its distal end through which the channel extends. The end piece can have an inner diameter that corresponds to the inner diameter of the hose or is also slightly smaller than the inner diameter of the hose. Preferably the electrode extends at least partly in the end piece such that the discharge base point is arranged shortly before the outlet opening inside the end piece.

In addition to the hydrophobic surface section formed on the electrode, additional hydrophobic surface sections can be provided. In doing so, capillary effects can be avoided. This means that on one hand an easier blowing out of liquid is allowed and the liquid absorption can be reduced due to the reduction of capillary effects. For this purpose, the end piece and/or the hose can comprise hydrophobic surface sections, particularly at the inner side adjacent to the channel, as necessary, however, also around the outlet opening and/or at the outer side of the end piece and/or the hose. With these measures the ignition readiness and steadiness of the probe can be supported remarkably.

In addition, it is possible to configure the electrode in a hollow manner, such that the electrode comprises a channel through which gas can flow. This electrode can be provided with a hydrophobic surface particularly in the proximity of its distal end or also completely on its outer side. It is also possible to provide the channel surrounded by the electrode with a hydrophobic surface.

In addition, it is possible to provide a flow guide element made of metal or an electrical insulator, such as plastic or ceramic, for support of the electrode in the channel and/or for influencing the gas flow, whereby the flow guide element can be configured to reduce the gas flow in the center of the cross-section, i.e. in proximity of the electrode, compared with the peripheral areas of the cross-section. Also this measure serves to increase the ignition readiness. The flow guide element can also be provided with a hydrophobic surface and can be provided with a hydrophobic surface structure or coating for this purpose.

In order to hydrophobically configure the desired surface sections, these surface sections can be provided with a hydrophobic coating, e.g. silicone oil, fluoropolymers, such as for example polytetrafluorethylene, parylene, PFA (perfluoroalkoxy), polyamide, polyethylene, a silicone elastomer or the like. Hydrophobic coatings can be applied, for example by a PVD or CVD process. In addition or as an alternative, it is possible to achieve the water-repellant effect of the respective surface sections by micro-structuring of the surface. For this, the respective surface sections can be provided with a plurality of intersecting finest grooves that spare a pattern of tiny projections that keep liquid droplets away from the groove bottoms with their faces, such that liquid can drip off easily.

### BRIEF DESCRIPTION OF THE DRAWINGS

Further details of preferred embodiments of the invention are subject of dependent claims, as well as the drawings of the description. The drawings show:

FIG. 1 a medical apparatus with a plasma probe attached thereto in a schematic illustration,

FIG. 2 the plasma probe of FIG. 1 in a longitudinal sectional illustration of its distal end section,

FIG. 3A-3D different embodiments of the electrode of the plasma probe according to FIG. 2 in longitudinal sectional enlarged illustration of its distal end,

FIG. 4 a modified embodiment of the plasma probe in longitudinal sectional illustration of its distal end,

FIG. 5 another embodiment of the plasma probe in longitudinal sectional illustration of its distal end,

FIG. 6 the plasma probe of FIG. 5 with view on the distal end,

FIGS. 7 and 8 additional embodiments of plasma probes in longitudinal sectional illustration of their distal ends respectively,

FIG. 9 the plasma probe according to FIG. 8 illustrating the flow profile,

FIG. 10 an embodiment of the plasma probe in longitudinal sectional illustration of its distal end.

### DETAILED DESCRIPTION

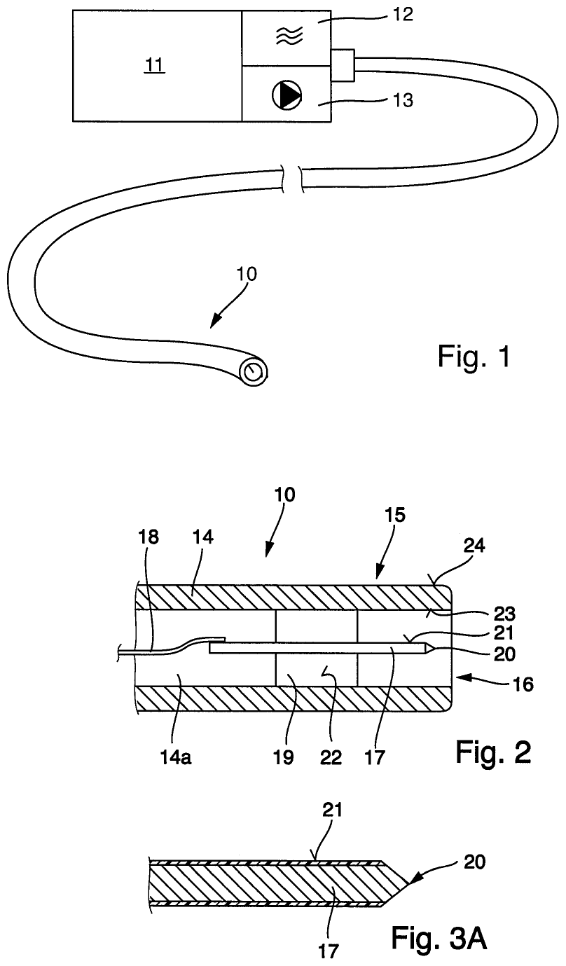

FIG. 1 illustrates a plasma probe 10 as it can be used in a not further illustrated endoscope for execution of a plasma treatment on a patient. For this the plasma probe 10 is connected to a supplying apparatus 11 or an apparatus arrangement that serves for supply of the plasma probe 10 with electrical current and an inert gas. For this the apparatus comprises an electrical generator, e.g. an RF generator 12 that is configured to output a radio frequency alternating voltage that is sufficient to ignite and maintain a plasma discharge.

A gas source 13 that supplies a suitable gas, e.g. argon, serves for supply with inert gas. The gas source 13 is thereby configured to supply gas in a dosed and controlled manner in order to supply an appropriate volume flow of gas to the plasma probe 10.

The plasma probe 10 is connected with the RF generator 12 and the gas source 13 at its proximal end, e.g. via a suitable connector.

The plasma probe 10 consists substantially of a flexible fluid line 14, e.g. in the form of a plastic hose. It can consist of PTFE or another plastic suitable for medical applications. The fluid line 14 surrounds a channel 14a that leads to the outlet opening 16 provided at the distal end 15 of the hose 14. An electrode 17 is arranged in the distal end 15, for example in the form of a metallic pin pointed at the end or a wire. While the distal end of the electrode 17 is arranged inside the channel 14a preferably approximately centered, such that it does not project from the outlet opening 16, the proximal end of the electrode 17 is connected with a suitable electrical line 18 that leads to the generator 12.

For positioning the electrode 17 in the channel 14a, a suitable holding device can be provided, e.g. a plate-shaped holder 19 positioned diametrically in the channel 14a that is supported with both longitudinal edges on the inside wall of the hose 14. The electrode 17 can be clamped, welded or otherwise connected with the holder 19. It is also possible to configure the electrode 17 and the holder 19 as one single part, e.g. as rhombic sheet metal part, the tip of which takes the position of the tip 20 of the electrode 17 and the longitudinal edges of which are supported on the inner side of the hose 14.

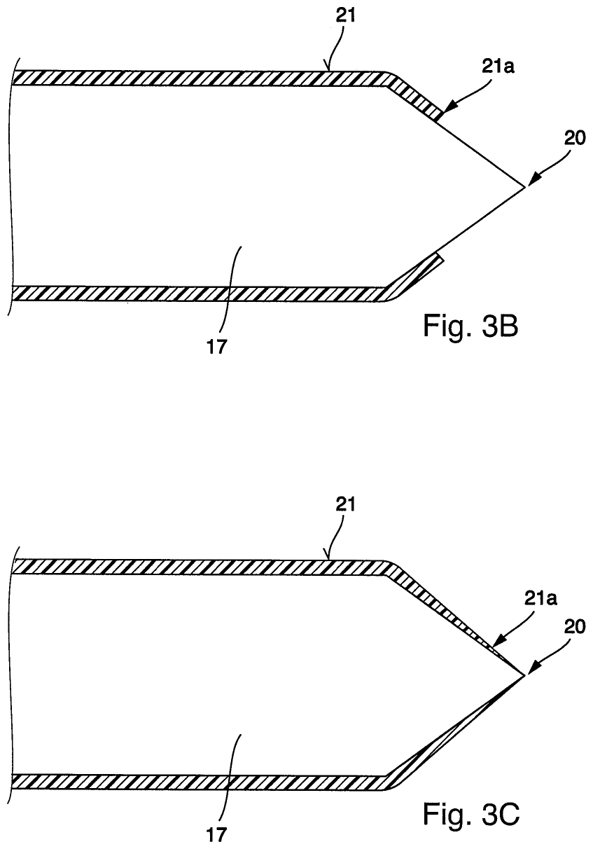

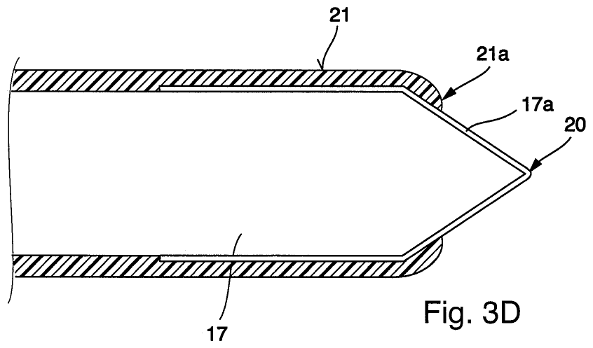

According to one aspect of the invention, the electrode 17 comprises at least one hydrophobic surface section 21. It preferably surrounds at least the distal end of the electrode 17. The hydrophobic surface section 21 can particularly cover the cylindrical outer peripheral surface of the electrode 17. FIG. 3A illustrates the hydrophobic surface section 21 that can be created by a water-repellant coating of the electrode 17. The electrode 17 consisting, e.g. of tungsten or another metal is provided with a layer of water-repellant material at its outer peripheral surface, e.g. of PTFE, silicone oil or the like. Preferably the tip 20 or a section thereof is, however, left blank, i.e. uncoated, whereby the expression “tip” means the total tapering, e.g. conically shaped section of the electrode 17.

The electrode 17 can also be provided with a coating extending into the region of the tip that forms the hydrophobic surface section 21. Thereby the coating can extend with a non-reducing thickness up to its end 21a or, as illustrated in FIG. 3C, can extend toward its end 21a in a manner reducing the thickness continuously or in steps. In addition, each of the electrodes 17 described above can support an electrically well-conducting layer 17a, e.g. a layer of silver, as illustrated in FIG. 3D by way of example of electrode according to FIG. 3B. This layer 17a is covered by the coating of the hydrophobic surface section 21 up to the tip or into the area of the tip 20. The electrode 17 itself can consist of stainless steel, steel, tungsten, hard metal or another refractory material.

The water-repellant effect in the hydrophobic surface section 21 can be obtained by the low surface energy of the used material that is preferably less than 20 mJ/m². In addition or as an alternative, the hydrophobic effect in the surface section 21 can be achieved by micro-structuring of the surface. For this purpose the surface section 21 is formed such that a multiplicity of very small peak-like projections project radially therefrom. For example, this can be achieved in that the surface section 21 is first provided with a helical groove and subsequently provided with axial grooves, e.g. by means of a laser. The length of the remaining projections and their distances to one another are thereby adjusted in a coordinated manner, such that a water droplet placed on the tips of these projections does not get into contact with the bottom of the groove with the meniscus formed between the projections due to its own surface tension. Also other methods for micro-structuring or influencing of the surface roughness can be applied, such as dry etching or the application of nano particles.

The tip 20 can be hydrophobic due to its large curvature of its conical surface and its tip.

In addition, the holder 19 can have a hydrophobic surface section 22 that covers the surface of the holder 19 completely or partly. Again the hydrophobic surface section 22 can be obtained by suitable micro-structuring of the surface or by coating thereof with water-repellant material. The above description with reference to the electrode 17 and the hydrophobic surface section 21 applies accordingly for the structuring of the surface or to the coating materials.

In addition, the hose 14 can be configured in a hydrophobic manner at the inside and/or at the outside and comprise respective hydrophobic surface sections 23, 24. Similarly its face can be hydrophobic. This can be achieved by suitable material selection, e.g. in that the hose 14 consists of PTFE. The hydrophobic effect of its surface can be increased by micro-structuring and/or coating or material selection as explained above.

The plasma probe 10 described so far operates as follows:

During operation a gas flow, e.g. an argon flow, having a defined amount, flows through the channel 14a. For example, a radio frequency alternating voltage is applied to the electrode 17. The patient is connected to a neutral electrode. Originating from the tip 20 a plasma stream is formed that hits the tissue.

During interruption of operation or prior to the start of operation, liquid, e.g. water, body liquid, flushing liquid or the like, can reach the outlet opening 16. However, the hydrophobic surface sections 21 and also 22, 23, 24 avoid the ingress of liquids in the channel 14a or at least the sticking of liquid droplets in the channel 14a and on the electrode 17. Already a very low gas flow is thus sufficient to free the electrode 17 and the channel 14a from ingressed liquid. The electrode 17 that remained dry in this manner can easily ignite with large distance to the tissue. A gas stream that is maintained low thereby supports the ignition readiness.

The same applies during introduction of the plasma probe 10 through an endoscope in the patient prior to the start of the treatment. Also in this case liquid may proceed into the channel 14a that can be flushed away from the electrode 17 already with very low gas flows.

Numerous modifications can be made to the invention that was thus far explained in general:

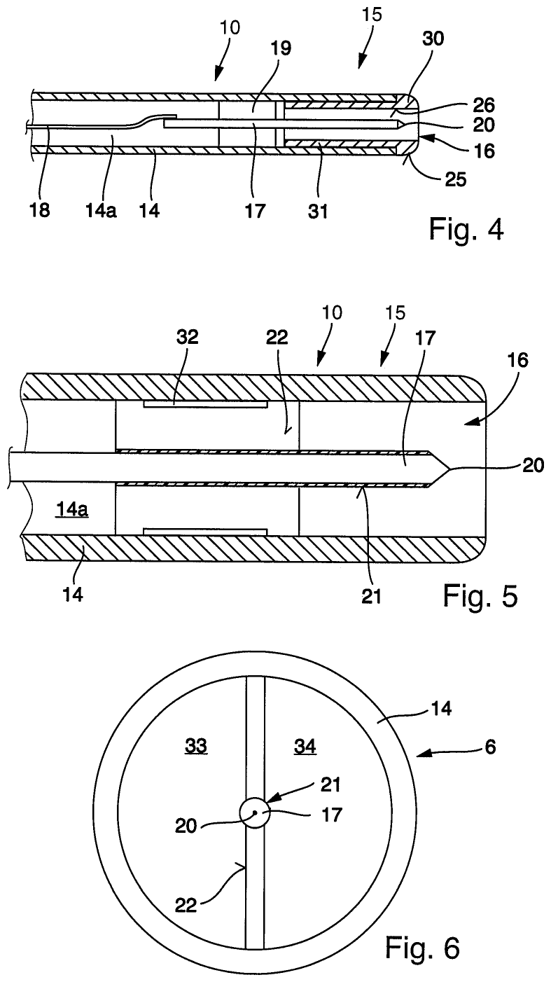

FIG. 4 illustrates a modified plasma probe 10 that comprises in addition a ceramic insert 30 in the distal end 15 of the hose 14 compared with the plasma probe 10 according to FIG. 2. This insert 30 comprises an elongated shank 31 configured in a hollow manner that is inserted into the hose 14. The channel 14a extends through the hose 14 and the shank 31 up to the outlet opening 16. The tip 20 of the electrode 17 is arranged inside the insert 30, preferably in the proximity of the outlet opening 16. The shank 31 serves for heat distribution and dissipation. The insert 30 can be provided with a hydrophobic surface section 25 on its outer side in the range of its distal end. Similarly it can comprise a hydrophobic surface section 26 at the inner side on its wall. The water-repellant effect of the surface sections 25, 26 can be achieved by each of the above-described ways due to appropriate material selection and/or structuring of the surface. Apart therefrom, the description provided with reference to FIGS. 1 and 2 applies accordingly.

The plasma probe 10 according to FIG. 2 is again illustrated in FIGS. 5 and 6 individually. The holder 19 can be configured as cooling element and can have one or more recesses at its hose side edges that minimize the contact surface to the hose. The holder 19 can be made of a metal, a ceramic material or a plastic material or an elastomer, such as for example silicone. Flow channels 33, 34 are provided on both sides of the holder 19, as shown in FIG. 6, through which gas, e.g. argon, can flow toward the open hose end. FIG. 9 illustrates the exiting gas flow 35 and the plasma that forms therein.

Again, as shown in FIG. 5, the electrode 17 can comprise a hydrophobic surface section 21 that extends preferably from the tip 20 up to the holder 19. Also the holder 19 can comprise hydrophobic surface sections 27, 28 on its face and/or in the flow channels 33, 34. The surface sections 27, 28 can be hydrophobic, because the holder 19 itself consists of hydrophobic material, because it is covered with hydrophobic material and/or because its surface is micro-structured to provide a lotus effect. With regard to the micro-structuring, the explanation provided in the context with the electrode 17 applies accordingly. Apart therefrom, the above description with reference to FIGS. 1-4 applies in a supplemental manner also for the embodiment according to FIGS. 5 and 6.

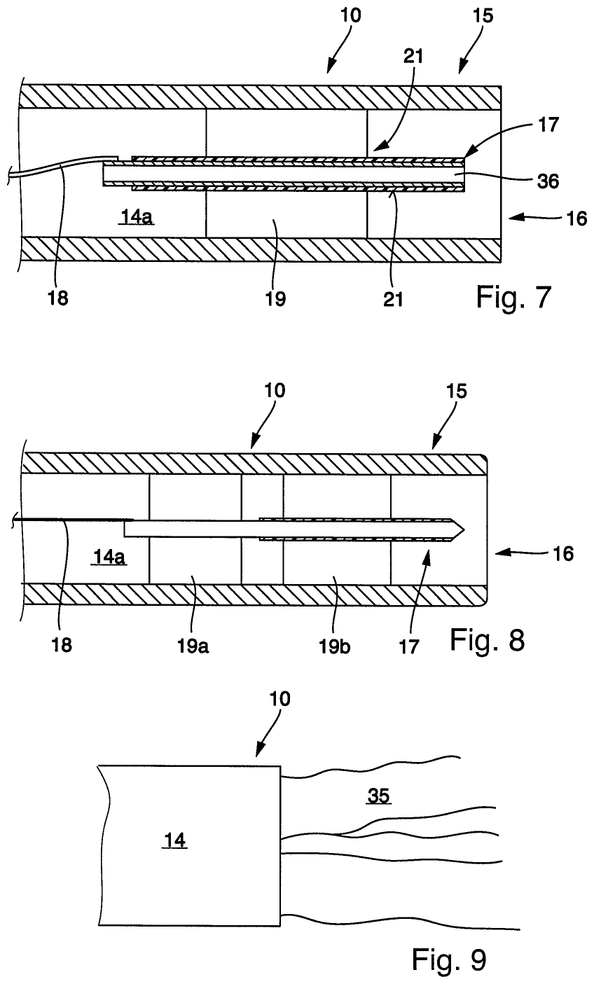

A further modified embodiment of the plasma probe 10 is illustrated in FIG. 7. For this embodiment first the description of the plasma probe 10 according to FIGS. 5 and 6 applies. Different thereto the electrode 17 is, however, hollow and thus configured without tip. It surrounds a narrow channel 36. The electrode 17 that is thus configured as small tube, is again connected with the wire 18 or another conductor. Its distal end does not project through the outlet opening 16. On its outer side the electrode 17 carries, for example, again the hydrophobic coating. The surface section 21 can be hydrophobic, because the electrode 17 itself consists of hydrophobic material, because it is coated with hydrophobic material and/or because its surface is micro-structured to provide a lotus effect.

A further modification of the plasma probe 10 is apparent from FIG. 8. The plasma probe 10 illustrated there comprises two holders 19a, 19b for supporting the electrode 17 that extend across the lumen of the hose and can be arranged in a manner turned with reference to each other around the electrode 17. Each of the indicated elements, particularly the electrode 17 as well as one of the holders 19a, 19b or also both holders can have a hydrophobic surface and for this purpose can consist of hydrophobic material or can be configured in a hydrophobic manner by a coating with hydrophobic material or by a surface structuring.

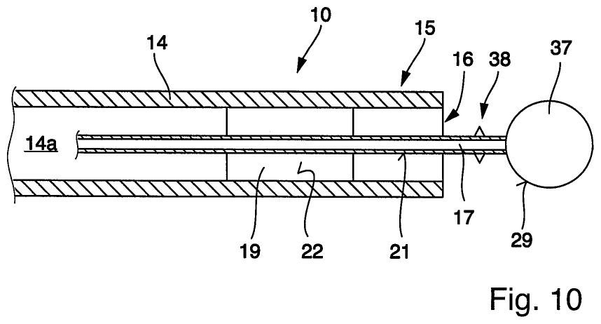

The electrode 17 must not necessarily be in a retracted position behind the outlet opening 16 inside the channel 14a. It is also possible, as apparent from the plasma probe 10 according to FIG. 10, to extend the electrode 17 out of the outlet opening 16. In this case, the electrode 17 is preferably provided with a blunt, preferably insulating, body 37, e.g. a ceramic ball, that avoids a direct galvanic contact between the electrode 17 and the surrounding biological tissue as well as mechanical harm of the tissue by the electrode 17. The electrode 17 can be supported by a holder 19, as illustrated in FIG. 10 or also by multiple holders similar to FIG. 8. The electrode 17 can have a tip 38 as discharge base point arranged in front of the outlet opening 16 and extending radially away from the electrode 17. The electrode 17 can again comprise a hydrophobic surface section 21. A further hydrophobic surface section 29 can be formed on the insulator 37 and can cover it completely or partly. The surface section 29 can be hydrophobic, because the insulator 37 itself consists of hydrophobic material, because it is coated with hydrophobic material and/or because its surface is micro-structured to provide a lotus effect. Apart therefrom, the above descriptions apply accordingly, particularly with regard to the configuration of hydrophobic surface sections 21 to 27.

In all embodiments according to FIGS. 5 to 10 a hollow cylindrical insert for thermal protection of the hose 14 can be inserted in the distal end 15 of the hose 14. The inner diameter of this insert can correspond to the inner diameter of the channel 14a or can also be slightly smaller than that. The insert can be hydrophobic, particularly on its inner wall and/or on at least one of its faces. For this the insert itself can consist of hydrophobic material, can be coated with hydrophobic material and/or can be micro-structured on the surface in order to provide a lotus effect.

A plasma probe 10 according to one aspect of the invention comprises a fluid line 14 that is preferably configured as hose and an electrode 17 arranged therein, the tip 20 of which is placed in proximity to the outlet opening 16. The electrode 17 comprises a surface section 21 that is configured hydrophobically. As an option, additional elements of the plasma probe 10, particularly in proximity of the outlet opening 16, can be provided with hydrophobic surfaces. Such a probe comprises a remarkably improved ignition readiness and due to the permanence of the resulting discharge base point an improved steadiness.

### LIST OF REFERENCE SIGNS

10 plasma probe
11 apparatus
12 RF generator
13 gas source
14 fluid line (plastic hose)
14a channel
15 distal end of fluid line
16 outlet opening
17 electrode
18 line
19 holder
20 tip of electrode 17
21-29 hydrophobic surface section
21a end of hydrophobic surface section
30 insert
31 shank
32 recess
33 flow channel
34a flow channel
35 gas flow
36 channel
37 insulator
38 tips

1. A plasma probe (10) for plasma coagulation of biological tissue, comprising:
a fluid line (14) having at least one channel (14a) configured to be connected to a gas source (13), wherein the at least one channel (14a) leads to a distal end (15) of the fluid line (14) and terminates at an outlet opening (16) at the distal end; and
an electrode (17) in or proximate to the outlet opening (16) configured to be connected to an electrical source (12) via a line (18); wherein the electrode (17) includes a hydrophobic surface section (21).

2. The plasma probe according to claim 1, wherein the fluid line (14) is a flexible hose made of electrically insulating material, and an electrically insulating heat-resistant end piece (30) is disposed in the distal end (15) of the fluid line (14), wherein the at least one channel (14a) extends through the electrically insulating heat-resistant end piece (30).

3. The plasma probe according to claim 1, wherein at least a portion of the electrode (17) is disposed within the electrically insulating heat-resistant end piece (30).

4. The plasma probe according to claim 2, wherein at least one of the end piece (30) or the flexible hose (14) includes a hydrophobic surface section (26).

5. The plasma probe according to claim 1, wherein the electrode (17) is hollow and comprises a hydrophobic surface section (21) on an outer side thereof.

6. The plasma probe according to claim 1, wherein a holder (19) is arranged at or adjacent to the distal end (15), wherein the holder (19) is connected with the electrode (17) for positioning the electrode (17) in the fluid line (14).

7. The plasma probe according to claim 6, wherein the holder (19) includes a hydrophobic surface section (27, 28).

8. The plasma probe according to claim 1, wherein the hydrophobic surface section (21) has a wetting contact angle (α) with water that is larger than 90°.

9. The plasma probe according to claim 1, wherein a surface energy of the hydrophobic surface section (21) is less than 20 mJ/m².

10. The plasma probe according to claim 1, wherein the hydrophobic surface section (21) is formed by a hydrophobic material.

11. The plasma probe according to claim 6, further comprising at least two channels (33, 34) extending along the holder (19).
